(19) United States
(12) Patent Application Publication
(10) Pub. No.: US 2003/0094506 A1
(43) Pub. Date: May 22, 2003

(54) Refill emanator for an air freshener or insecticide dispenser device

(76) Inventors: Shaun Patrick Rymer, Beverley (GB); Clare Louise Robinson, Hull (GB); Andrew Robert McLeish, Sheffield (GB)

Correspondence Address: FISH & RICHARDSON P.C., 45 ROCKEFELLER PLAZA, SUITE 2800, NEW YORK, NY, 10111, US

(21) Appl. No.: 09/989,294

(22) Filed: Nov. 20, 2001

(51) Int. Cl. A61L 9/04

(52) U.S. Cl. 239/43; 239/145

(57) ABSTRACT

A refill for a liquid air freshener or insecticidal device comprising a cartridge containing a volatile liquid composition to be evaporated and a porous pad positioned, in use, beneath a lower face of the cartridge to receive the liquid composition as it is slowly released from the cartridge; wherein the porous pad is a part of a sheet of material which is folded to at least partially surround the cartridge.

The present invention relates to a refill for a liquid air freshener or insecticidal device.

Refills are known in the art which comprise a cartridge containing a volatile liquid composition to be evaporated and a porous pad positioned, in use, beneath a lower face of the cartridge to receive the liquid composition as it is slowly released from the cartridge.

The present invention provides an improvement to such a refill. According to the present invention, the porous pad is a part of a sheet of material which is folded to at least partially surround the cartridge.

Such an arrangement provides a simple way of securing the porous pad reliably in position beneath the cartridge which does not require a separate fitting operation by the consumer. It also provides a more substantial package which can be easily handled by the consumer and readily located with the housing of the air freshener or insecticidal device.

The folded sheet may be fixed directly to the cartridge. However, preferably, a frame of rigid material is attached to the cartridge and the sheet of material is attached to the frame. This provides a convenient-way of attaching the sheet firmly to the cartridge and provides a more rigid structure which is easier for the consumer to handle.

The frame and sheet are preferably provided as a sub-assembly wherein the frame has a foldable configuration with at least some of the fold lines corresponding to fold lines on the sheet.

Preferably, the sheet of material comprises the porous pad, a rear panel and two side panels which are folded inwardly towards one another from opposite sides of the rear panel. The porous pad may either be foldably attached to a bottom edge of the rear panel, or may be attached to one or both bottom edges of the side panels.

Two examples of refills constructed in accordance with the present invention will now be described with reference to the accompanying drawings, in which:

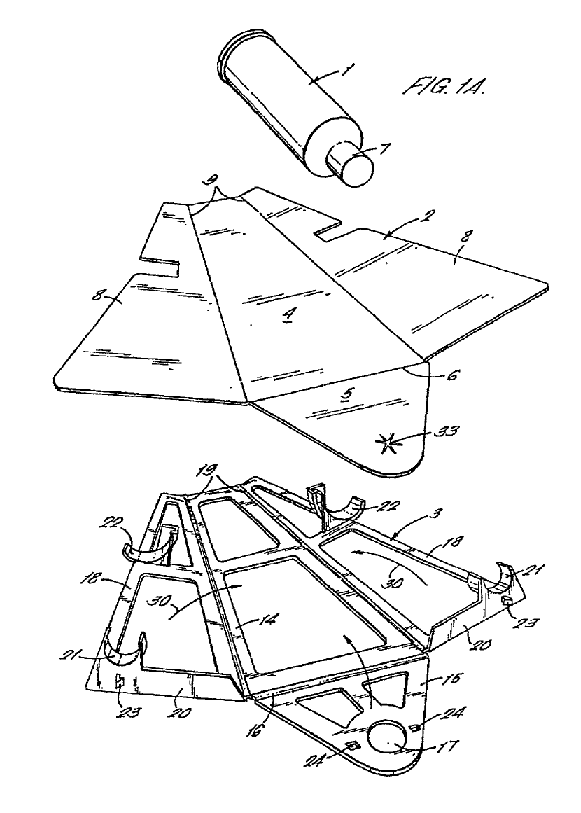

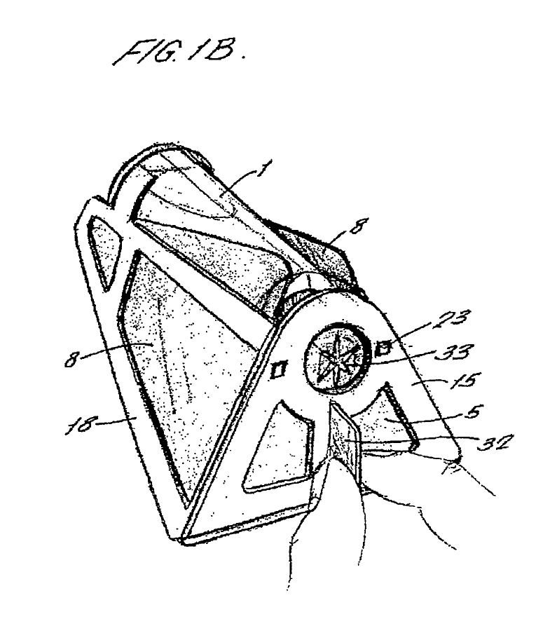

FIG. 1A is an exploded view of a first example showing the assembly of the refill;

FIG. 1B is a perspective view of the assembled refill;

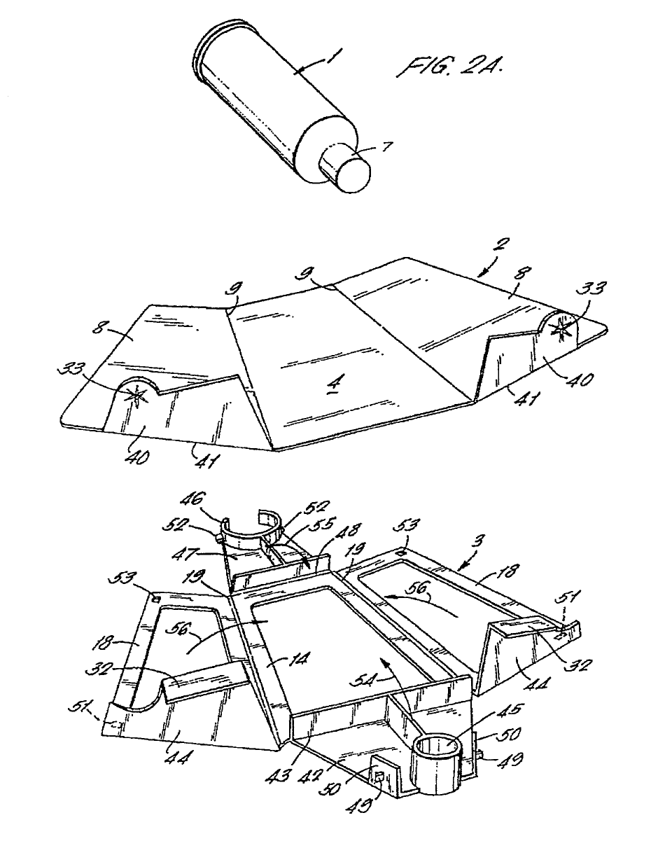

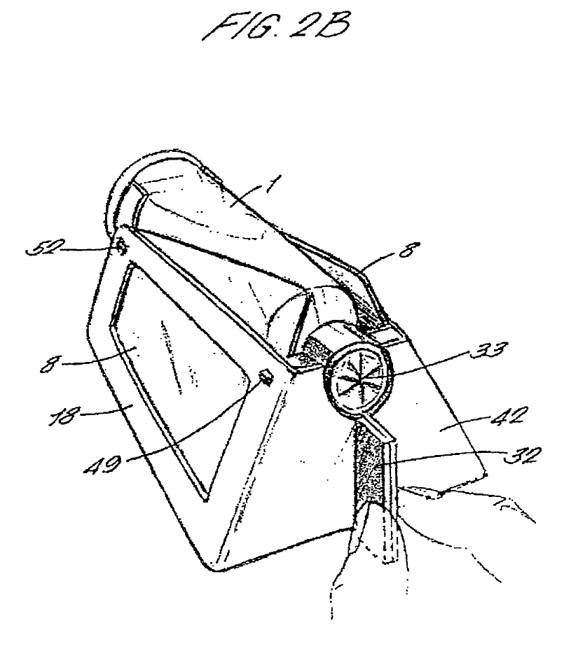

FIG. 2A is a view similar to FIG. 1A of a second example; and

FIG. 2B is a view similar to FIG. 1B of a second example.

The first example will now be described with reference to FIGS. 1A and 1B.

The refill comprises three components, a cartridge 1, a cardboard sheet 2, and a plastic frame 3.

The sheet 2 comprises a rear panel 4 with a base panel 5 attached along a fold line 6 to its lowermost edge. The base panel 5 provides the porous pad onto which, in use, liquid composition is gradually dispensed from the lower end 7 of cartridge 1. Two sheet side panels 8 are attached via fold lines 9 along the side edges of the rear panel 4.

The sheet 2 sits on the frame 3 which has a shape which generally conforms to the shape of the sheet 2. Thus, the frame 3 has a rear panel 14 corresponding in shape to the rear panel 4 of the sheet and a base panel 15 corresponding in shape to the base panel 5 of the sheet and attached to the rear panel 14 of the base along a fold line 16. The base panel 15 has an aperture 17 positioned to allow penetration of the lower end 7 of the cartridge 1. Two side panels 18 are attached along opposite sides of the rear panel 14 along fold lines 19. An upstanding flange 20 projects from the bottom edge of each side panel 18. A cartridge engaging jaw member 21 projects from the outer edge of the upstanding flange 20. A second jaw member 22 is provided towards the top of each side panel 18. A clip 23 extends from a downwardly facing surface of the upstanding flange 20. The clips 23 are arranged to engage in through holes 24 in the base panel 15.

To assemble the refill, the sheet 2 is positioned on the frame 3. The side panels 8/18 are then folded inwardly in the direction of arrows 30 until the cartridge 1 is held by jaw members 21 and 22. The base panel 5/15 is then folded upwardly in the direction of arrow 31 so that the clips 23 clip into the holes 24, and so that the lower end 7 of cartridge 1 is aligned with the aperture 17, thereby forming the finished refill as shown in FIG. 1B. The clips 23 penetrate the base panel 5 to assist in securing the sheet 2 in place.

As shown in FIG. 1B, a tab 32 is provided on the lower surface of the base panel 15 to allow the refill to be removed from the device housing. Further, a number of slits form a star-shaped aperture 33 in the base panel 5. This aperture 33 is arranged to be pierced by a spike in the housing into which the refill is placed. The spike also pierces the lower end 7 of the cartridge 1 to allow flow of the composition out of the cartridge 1 thereby initiating operation of the device.

A second example of the invention will now be described with reference to FIGS. 2A and 2B. Broadly, this has the same construction as the first example with the cartridge 1, cardboard sheet 2 and plastic frame 3. The sheet 2 has a similar rear panel 4 and two adjacent side panels 8 joined to the rear panel along fold lines 9. The frame 3 has a similar rear panel 14 and adjacent side panels 18 joined to the rear panel along fold lines 19.

However, in place of the base panel 5 of the first example, the sheet 2 is provided with a pair of base panels 40, each base panel being attached along a fold line 41 to a lowermost edge of a respective side panel 8. The base panels 40 are half the size of the base panel 5 of the first example and are arranged to meet one another when the refill is folded up to provide a similar configuration to the base panel 5 of the first example. Each base panel 40 has a portion with the star-shaped orifice 33. In the assembled refill, these portions will overlap forming a porous pad of double thickness beneath the lower end 7 of the cartridge 1.

The frame 3 has a base panel 42 attached to the lowermost edge of the rear panel 14 via a fold line 43. Additional parts of the base are provided by a pair of half panels 44 which extend from the lowermost edges of respective side panels 18 and are arranged to support the base panels 40 of the sheet 2. Each half panel 44 has a tab 32 positioned so that tabs 32 abut one another in the assembled refill as shown in FIG. 2B.

The jaw member 21 of the first example is replaced by a cylindrical orifice 45 which is open at its bottom end as shown in FIG. 2B to allow the spike to penetrate when the refill is inserted into the housing. The upper jaw members 22 of the first example are replaced by a single jaw member 46 which is a part circular jaw member attached to the top of an upper panel 47 which is attached to the upper edge of the rear panel 14 along a fold line 48.

To clip the frame together, a pair of clips 49 are provided on flanges 50 which extend upwardly from an outer edge of the base panel 42. These engage with corresponding openings 51 in the side panels 18. A second pair of clips 52 are provided on the top panel 47. These engage with a second pair of openings 53 in the side panels 18.

With the sheet 3 in place on the frame 3, the base panel 42 is folded up in the direction of arrow 54 so that the bottom end of cartridge 1 is retained in cylindrical orifice 45. The top panel 47 is folded downwardly in the direction of arrow 55 so that the top of the cartridge 1 is retained within jaw member 46. The side panels 18 are then folded inwardly in the direction of arrows 56 bringing the tabs 32 together, and bringing the star-shaped orifices 33 into overlapping relation beneath the lower end 7 of the cartridge 1. At the same time, the clips 49 and 52 clip into their respective holes 51 and 53 securing the frame in place. The clips penetrate through the cardboard sheet 2 and also serve to secure this in place. The assembled refill can then be handled as described with reference to the first example.

We claim

1. A refill for a liquid air freshener or insecticide device comprising a cartridge containing a volatile liquid composition to be evaporated and a porous pad positioned, in use, beneath a lower face of the cartridge to receive the liquid composition as it is slowly released from the cartridge, wherein the porous pad is a part of a sheet of material which is folded to at least partially surround the cartridge.

2. A refill according to claim 1, wherein a frame of rigid material is attached to the cartridge and the sheet of material is attached to the frame.

3. A refill according to claim 2, wherein the frame and sheet are provided as a sub-assembly in which the frame has a foldable configuration with at least some of the fold lines corresponding to fold lines on the sheet.

4. A refill according to claims 1, 2 or 3, wherein the sheet of material comprises the porous pad, a rear panel and two side panels which are folded inwardly towards one another from opposite sides of the rear panel.

5. A refill according to claim 4, wherein the porous pad is foldably attached to a bottom edge of the rear panel.

6. A refill according to claim 4, wherein the porous pad is attached to one or both bottom edges of the side panels.

7. An air freshener or insecticide device having a housing adapted to accommodate a refill according to claim 1.

8. An air freshener or insecticide device having a housing adapted to accommodate a refill according to claim 4.

9. An air freshener or insecticide which comprises a housing containing a refill according to claim 1.

10. An air freshener or insecticide device which comprises a housing containing a refill according to claim 4.
